(19) United States
(12) Patent Application Publication
(10) Pub. No.: US 2013/0224850 A1
(43) Pub. Date: Aug. 29, 2013

(54) Device and method for detecting bacterial endospores that are suspended in the atmosphere

(71) Applicants: Ronald E. Meyers, Columbia, MD (US); David L. Rosen, Rockville, MD (US)

(72) Inventors: Ronald E. Meyers, Columbia, MD (US); David L. Rosen, Rockville, MD (US)

(21) Appl. No.: 11/723,438

(22) Filed: Mar. 20, 2007

Related U.S. Application Data
(63) Continuation-in-part of application No. 11/100,235, filed on Apr. 1, 2005, now abandoned.

(51) Int. Cl. C12M 1/42

(52) U.S. Cl. 435/288.7

(57) ABSTRACT

A device that detects bacterial endospores suspended in the atmosphere is provided. The device having an aerosol concentrator, a collection vessel containing a lanthanide salt solution, an excitation energy source and an optical set-up that directs the excitation energy source to the lanthanide salt solution and collects photoluminescence generated by the lanthanide salt solution upon receipt of the excitation energy source. A system and method for detecting bacterial endospores is also disclosed.

### CROSS-REFERENCE TO RELATED APPLICATIONS

This application is a continuation-in-part of U.S. patent application Ser. No. 11/100,235 filed Apr. 1, 2005 entitled Method And Composition For Bacterial Endospore Detection, the contents of which are incorporated herein by reference.

### GOVERNMENT INTEREST

The invention described herein may be manufactured, used, and licensed by or for the United States Government.

### BACKGROUND OF THE INVENTION

1. Field of the Invention

This invention relates generally to particle detection and, more particularly, to detecting the presence of bacterial endospores in the atmosphere.

2. Description of the Related Art

Unfortunately, living with the threat of a terrorist attack has become a way of life in the world today. As a result, the need to be ever vigilant has increased thereby creating the demand for new ways of detecting various forms of terrorist attacks.

While terrorist attacks can take place in many forms, one of the most deadly attacks would be in the form of a chemical or biological agent dispersed through the air. A terrorist attack resulting from the dispersal of an agent such as anthrax in the air over a city or within an office building could have a devastating effect as many people could become affected by the anthrax without their knowledge. Furthermore, given present ways of detection, responsible agencies and authorities would not even be aware of the attacks until the first symptoms began showing up in the attack victims; a time at which it is probably too late to save the majority of those exposed to the biological or chemical agent.

Anthrax and other biological warfare agents include spore-producing bacteria. An endospore is the dormant state of some genera of bacteria with a spore case that contains a large concentration of calcium dipicolinate (Ca(dpa)). When an endospore is placed in an appropriate solution, some of the calcium dipicolinate associated with the endospores will dissolve and release dipicolinate anions (dpa²⁻). However, bacterial endospore concentrations are not easily determined with conventional techniques and known tests for bacterial contamination are usually slow and often not reliable enough for common use. Serological methods use antibodies, which often have large cross reactivities that can cause false alarms. Mass spectroscopy has extremely complex spectra that are difficult to analyze and could also cause false alarms. DNA testing is extremely slow and expensive. In addition, microscopy and plate culture counting are both extremely slow and tedious to perform.

In contrast, a method of using lanthanide cations to react with dpa²⁻ from which photoluminescence can be measured has been developed. This method combines lanthanide cations with dpa²⁻ in a given solution and subsequently takes advantage of the long emission decay times and emission spectra of the lanthanide cations in order to provide photoluminescence information. Substances have luminescence lifetimes on the order of nanoseconds, whereas terbium dipicolinate and europium dipicolinate have relatively long luminescence lifetimes (2.0 milliseconds and 0.8 milliseconds, respectively). Therefore, the presence of any luminescence after several nanoseconds in an illuminated sample is highly indicative of the presence of lanthanide dipicolinate species and the endospores that are the source of dipicolinate ions. On the other hand, if no significant luminescence is present after several nanoseconds, the sample medium can be determined to contain no significant endospore content.

In order to ensure the luminescence is linear with dpa²⁻ concentration, a concentration of lanthanide, for example Tb³⁺, is chosen that is large enough to use or react with all the dpa²⁻ in the sample. However, the sensitivity of the endospore detection method is limited by the background luminescence of excess Tb³⁺, which does not react with dpa²⁻, since the emission spectra of Tb³⁺ and Tb(dpa)ₙ³⁻²ⁿ are essentially identical.

Therefore, to discriminate between Tb(dpa)ₙ³⁻²ⁿ from Tb³⁺, the temporal dynamics of the Tb(dpa)ₙ³⁻²ⁿ luminescence are determined.

Even with the ability to discriminate between Tb(dpa)ₙ³⁻²ⁿ from Tb³⁺, a practical method for collecting air in a given environment and sampling for endospores in a time-efficient manner is lacking. Given these shortcomings, a quick, accurate and low-cost device and method for detecting bacterial endospores in the atmosphere would be an important improvement in the detection of biological agents dispersed through the air.

### SUMMARY OF THE INVENTION

A device to detect bacterial endospores suspended in the atmosphere is provided. The device includes an active aerosol concentrator, a collection vessel containing a lanthanide salt solution, an excitation energy source and an optical set-up that directs the excitation energy source to the lanthanide salt solution and collects photoluminescence generated by the lanthanide salt solution upon receipt of the excitation energy.

A method for detecting bacterial endospores suspended in the atmosphere is also provided. The method comprises the steps of collecting a plurality of aerosol samples in an aerosol concentrator over a given period of time, illuminating the aerosol samples with a light source, collecting the photoluminescence emitted by the aerosol samples, and calculating the amplitude and lifetime of the photoluminescence.

### BRIEF DESCRIPTION OF THE DRAWINGS

FIG. 1 is a schematic showing one embodiment of a device where a broad band ultraviolet beam of radiation passes through a quartz collection vessel that contains a liquid solution of terbium chloride and an aperture of an integrating sphere is positioned to collect photoluminescence from terbium dipicolinate.

FIG. 2 is a schematic showing another embodiment where ultraviolet radiation transmitted through an optical fiber excites a liquid solution of terbium chloride inside a collection vessel and a second optical fiber collects the photoluminescence from terbium dipicolinate.

FIG. 3 is a schematic showing still another embodiment where a broad band ultraviolet beam of radiation passes through a quartz collection vessel that contains a sol-gel liquid that has a liquid phase containing terbium chloride and an aperture of an integrating sphere is positioned to collect photoluminescence from terbium dipicolinate.

FIG. 4 is a schematic showing yet another embodiment where ultraviolet radiation transmitted through an optical fiber excites a sol-gel with a liquid phase containing a dissolved terbium chloride and a second optical fiber collects the photoluminescence from the terbium dipicolinate.

FIG. 5 is a schematic showing a system for imaging a collected aerosol particle that contains bacterial endospores.

FIG. 6 is a schematic of one example of an optical set-up.

FIG. 7 is a graph showing the temporal profile of photoluminescence with a 1.14 ms delay after peak excitation using the device shown in FIG. 1. The graph depicts temporal profiles both before and after bacterial endospores were collected from an aerosol chamber.

FIG. 8 is a graph showing the amplitude for a temporal profile calculated after a 1.14 ms delay calculated assuming single exponent decay.

FIG. 9 is a graph showing a lifetime of emission for a temporal profile calculated after a 1.14 ms delay calculated assuming single exponent decay.

### DESCRIPTION OF THE PREFERRED EMBODIMENTS

A device that detects bacterial endospores suspended in the atmosphere is provided. The device is comprised of an aerosol concentrator containing a lanthanide salt solution that yields a photoluminescent lanthanide dipicolinate complex in the presence of bacterial endospores, an excitation energy source and an optical setup that directs the excitation energy source to the lanthanide salt solution and collects photoluminescence generated by the lanthanide salt solution upon receipt of the excitation energy source.

For purposes of the present invention, aerosol is defined as a suspension of fine solid or liquid particles that are suspended in a gas. An aerosol particle may or may not contain bacterial endospores. A water droplet suspended in the atmosphere is also an aerosol, whether or not it contains bacterial endospores. A cluster of soil particles suspended in the atmosphere is an aerosol particle whether or not it contains bacterial endospores. An isolated bacterial endospore suspended in the atmosphere is a type of aerosol particle. A cluster of bacteria stuck to together is a type of aerosol particle whether or not any of them are endospores. The inventive method of the present invention consists of collecting aerosol particles and determining, using the photoluminescence of a lanthanide dipicolinate complex, whether any of them either contain bacterial endospores or are bacterial endospores. The inventive device of the present invention includes all embodiments of this method.

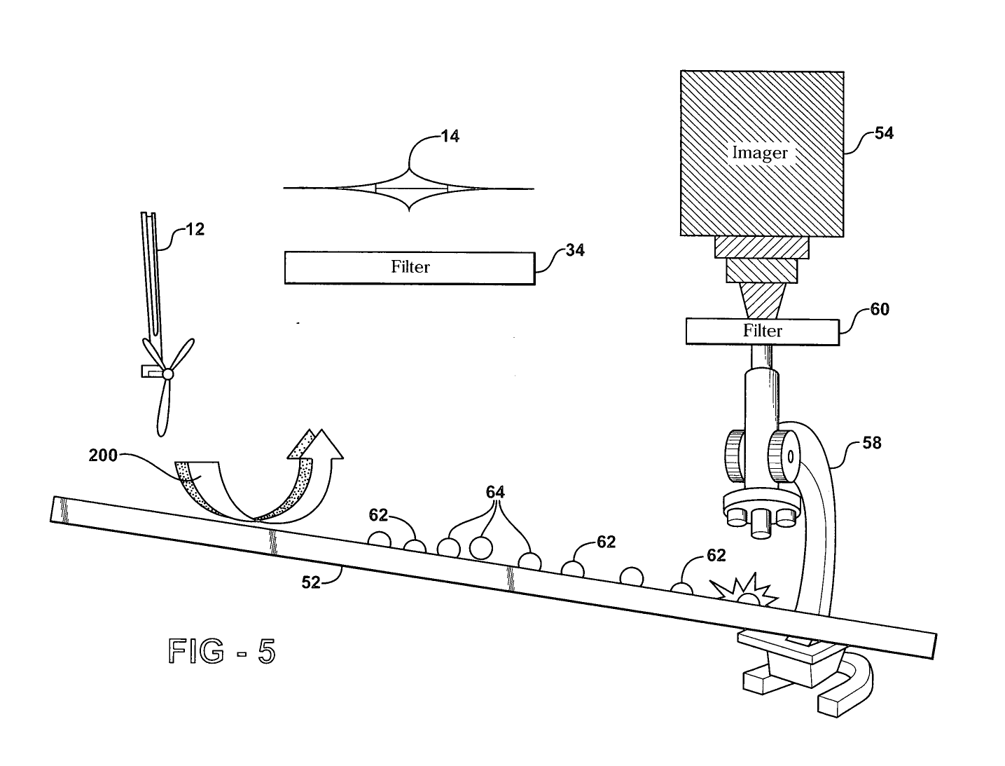

In one embodiment a system is provided that not only detects bacterial endospores suspended in the atmosphere, but also locates an aerosol particle that contains bacterial endospores. In this embodiment, shown in FIG. 5, the system has a container having a sol-gel with a lanthanide salt dissolved in a liquid phase, an impactor for depositing the aerosol particle on the sol-gel, a light source for transmitting an ultraviolet light through a broadband filter onto the sol-gel, and an imager.

The imager can help identify the type of particle that contains the endospore. For example, in FIG. 5, there could be two types of aerosol particle (62 and 64). By imaging the dipicolinate released by the endospore, together with the particle containing the endospore, the particle can be identified as being contained either in particle 62 or particle 64.

A method is also provided for detecting bacterial endospores suspended in the atmosphere. This method includes collecting a plurality of aerosol samples in an aerosol concentrator over a given period of time, illuminating the aerosol samples with a light source, collecting the photoluminescence emitted by the aerosol samples and calculating the amplitude and lifetime of the photoluminescence to determine at least whether endospores are present and optimally the quantity of spores present.

Emitted light is measured either during or after collection of the aerosols. Ultraviolet (UV) radiation illuminates the contents of a collection vessel such as a quartz container. The light emitted from the contents is collected by an optical device. Exemplary collection devices include a lens, an integrating sphere, or a fiber optic. The light may then pass through a dispersive device that selects a wavelength that is characteristic of a lanthide salt. An example of a lanthide is terbium or europium, an example of a dispersive device is a narrow band filter or a spectrometer, and an example of an emission band is a 542 nanometer (nm) band for terbium salts.

A photodetector measures the intensity of emission as a function of time before and after excitation. Examples of photodetectors include photomultipliers, photodiodes and charge coupled diode arrays. The signal from the photodetector is sent to a temporal analyzer which may be for example a digital oscilloscope, a multichannel analyzer, or an A/D converter coupled with a computer. The temporal analyzer analyzes the temporal file of the emitted light. An example of such an analysis is the determination of emission lifetime, the measurement of peak intensity, the measurement of time integrated intensity, or phase modulation.

Analysis of the photoluminescence can include both isolating the emission at one of the terbium band wavelengths (490, 542, 586 and 622 nm), measuring the time integrated emission after a time delay, or using an electronic filter to isolate the time resolved component with the same lifetime as terbium dipicolinate, which is on or about 0.6 ms.

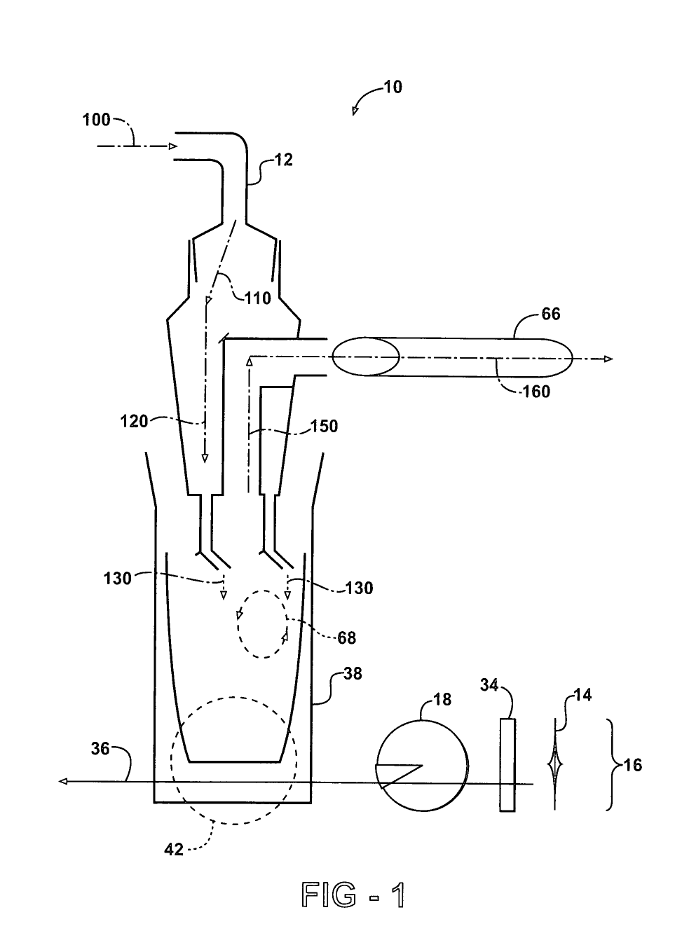

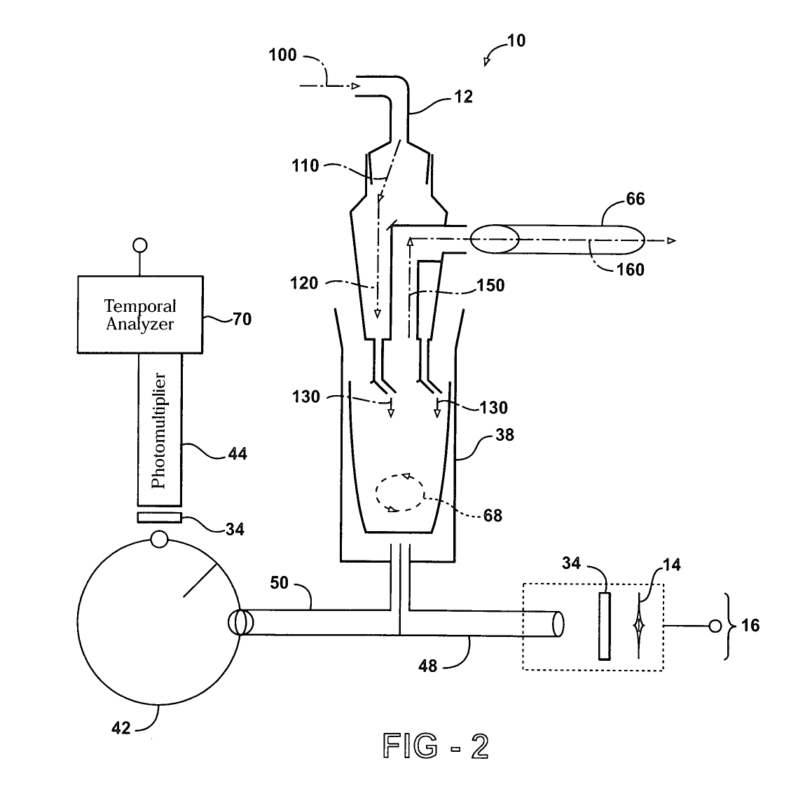

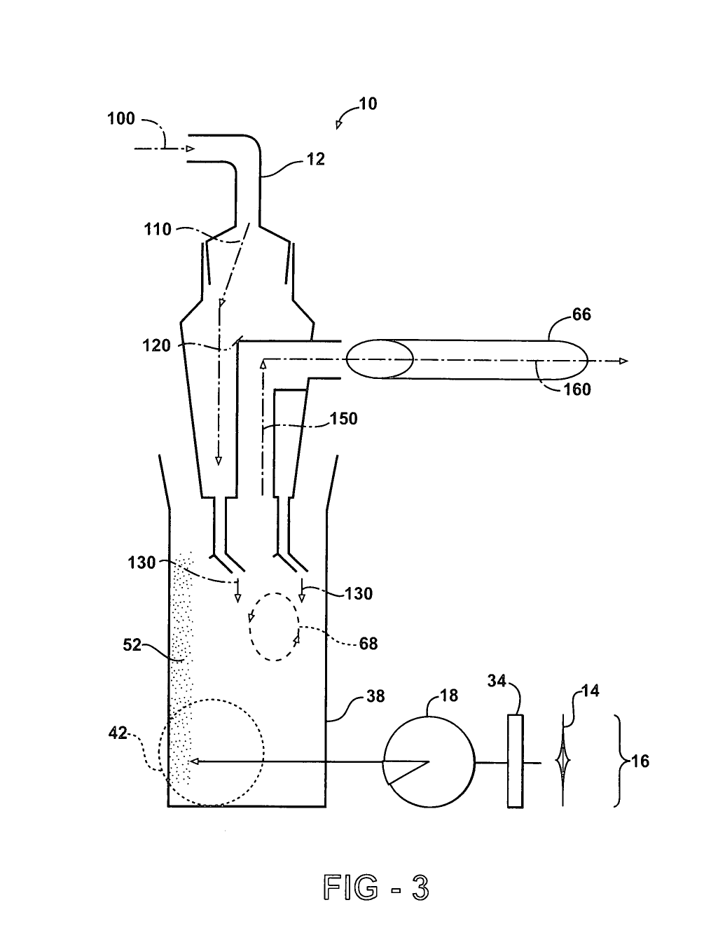

A device 10 to detect bacterial endospores suspended in the atmosphere is provided. Different embodiments of the device are shown in FIGS. 1-5. The device 10 includes an active aerosol concentrator 12, a collection vessel 38 containing a lanthanide salt solution. In the embodiments of FIGS. 1 and 2, the solution is a circulating liquid on the bottom and sides of the collection vessel 38. In the embodiments shown in FIGS. 3-5 the solution is embedded in a sol gel matrix 52 on the side of the collection vessel.

The lanthanide salt solution yields a photoluminescent lanthanide dipicolinate complex in the presence of bacterial endospores, and preferably includes a polar solvent or a room temperature ionic liquid. Also included in the device 10 is an excitation energy source 14 that provides excitation energy 36 and an optical set-up 16 that directs the excitation energy 36 to the lanthanide salt solution and collects photoluminescence generated by the lanthanide salt solution upon receipt of the excitation energy 36. In the alternative, the device 10 is comprised of an active aerosol concentrator. 12 containing a lanthanide salt solution within a collection vessel 38, and an optical set-up 16 that directs excitation energy 36 to the collection vessel 38 and subsequently collects photoluminescence generated by the lanthanide salt solution upon receipt of the excitation energy 36. The active aerosol concentrator 12 can selectively be one of several types including a bubbler, an impinger, an impactor and combinations thereof. Furthermore, the lanthanide salt solution can be one of various types including trivalent terbium cations or europium cations.

The active collection of a gas, e.g. air, with or without endospores contained therein, into the aerosol concentrator 12 is shown by arrow 100. The aerosol concentrator 12 is therefore an active concentrator. For purposes of the present invention an active aerosol concentrator is defined as a concentrator that affords for a forced gas flow in a desired direction and does not rely on particles to freefall onto a desired surface. Freefall is defined as a particle falling or descending due to the force of gravity alone. The gas travels through the concentrator 12 as illustrated by arrows 110 and 120 before exiting the aerosol collector as shown by arrows 130 and entering the collection vessel 38. The source of the forced gas flow can be any method or article known to those skilled in the art, illustratively including a fan that forces the gas in a desired direction, a pump that forces the gas in a desired direction, a draft that pulls the gas in a desired direction, a vacuum that pulls the gas in a desired direction and combinations thereof.

A bubbler aerosol concentrator forces the gas to bubble through a liquid, e.g. the lanthanide solution, whereas an impinger aerosol concentrator forces the gas and any included aerosol particles to impinge against a desired surface. The gas and any included aerosol particles are typically impinged against a desired surface by blowing the gas through an exit aperture of the aerosol concentrator 12. A desired surface can include any surface known to those skilled in the art, illustratively including a membrane 52 as shown in FIGS. 3-5. An impactor aerosol concentrator collects the aerosol particles, forces said particles into an air stream 200 created by a fan 12, and impacts the aerosol onto a desired surface 52 (FIG. 5).

As shown in FIGS. 1-5, the excitation energy source 14 may be an ultraviolet light, illustratively including a mercury (Hg) arc lamp, a light emitting diode, a fluorescent tube, a Hg vapor—metal halide lamp and combinations thereof. This or any other energy source 14 may be modulated through the use of a chopper, synonymously described herein as an electronic modulator 18. Furthermore, ultraviolet filters 34 are optionally used to filter the energy source 14. These filters 34 may include narrow or broadband pass filters.

FIG. 6 depicts one type of optical set-up or apparatus 16 that may selectively be used in concert with the present invention. In this apparatus, an aperture 20 is located proximate an ultraviolet light source 14. The aperture 20 affords a beam of light 36 from the light source 14 within the apparatus 16. A first lens 22 is positioned adjacent the aperture 20 downstream of the light source 14. A second lens 24 is located downstream from the first lens 22 such that the first and second lenses 22 and 24 together form a focusing element and optimally bracket a modulator 18. A third lens 26 forms a second focusing element, is positioned downstream of the second lens 24, and preferably with second lens 26 brackets a silica window 28. The silica window 28 can be partially mirrored and affords for the reflection of at least part of the beam of light 36 to a photodiode 32. A third focusing element shown as fourth lens 30 in FIG. 6 is located proximate the silica window 28 and the photodiode 32, said forth lens 30 focusing the reflected portion of the beam of light 36. An ultraviolet filter 34 is preferably bracketed between the fourth lens 30 and the photodiode 32. The filter 34 protects the photodiode 32 from ambient light.

The fourth lens 30 is positioned at a location greater than 0° but less than 180° relative to a beam of light 36 generated by the ultraviolet light source 14. A collection vessel 38 may selectively be located downstream of the third lens 26. The third lens 26 focuses the beam of light 36 after said beam passes through the silica window 28. The collection vessel 38 and the third lens 26 preferably bracket a second broadband ultraviolet filter 40. Similar to the ultraviolet filter 34 above, the filter 40 protects the collection vessel 38 from ambient light.

The optical apparatus 16 includes an integrating sphere 42 positioned adjacent the collection vessel 38 and a photomultiplier 44. In particular, a digital storage oscilloscope 46 is connected to both the photomultiplier 44 and the photodiode 32, with the photodiode 32 serving as a trigger for the oscilloscope 46.

When in operation, a lanthanide salt solution such as terbium chloride is placed in an active aerosol concentrator 12 as seen in FIGS. 1-4. In this particular example, a Bioaerosol® sampler manufactured by SKC, Inc. is used operating in its impinger mode, however, any suitable active aerosol concentrator 12 may be used. Aerosols are then collected. The detection method can be used continuously over many minutes and hours without stopping the aerosol collection process, turning off the UV excitation source, or resetting the background level of the detector. If the bacterial endospore concentration in the atmosphere increases suddenly over background level, the detector can rapidly detect the increase within seconds. By collecting aerosols for a longer period of time, the sensitivity of the detector may be increased. The temporal profile of the aerosols is measured using a photomultiplier 44 and analyzed with a temporal analyzer 70, as shown in FIG. 2. This procedure can then be repeated over a period of time, for example 41 minutes. In one embodiment of the present invention, the device is operable to collect aerosols automatically and/or by remote activation.

Other embodiments concern the form of the lanthanide solution. For example, the solvent can be water, glycerol, ethylene glycol, or any mixture thereof. As an alternative, the solvent can be a room temperature ionic liquid such as ethyl ammonium nitrate, a dialkylimidazolium salt, or any mixture thereof. The solution can be in a purely liquid phase, or embedded in a sol-gel. The optimal choice of lanthanide solution is determined by the conditions under which the sensor is used.

A method for detecting endospores is described in U.S. Pat. No. 5,876,960 (Rosen), the contents of which are incorporated herein by reference. In order to detect endospores in the atmosphere, the lanthanide solution is combined with the aerosols collected. If bacterial endospores are present in the aerosols, the lanthanide (e.g., terbium or europium) reacts with calcium dipicolinate from the endospores to produce a lanthanide chelate, specifically, terbium or europium dipicolinate. The lanthanide chelate has distinctive absorbance and emission spectrums (e.g. a long decay time) which are detected using the photodiode 32, photomultiplier 44 and combinations thereof. The detected absorbance and emission spectrums are analyzed using the digital storage oscilloscope 46, the temporal analyzer 70 and combinations thereof.

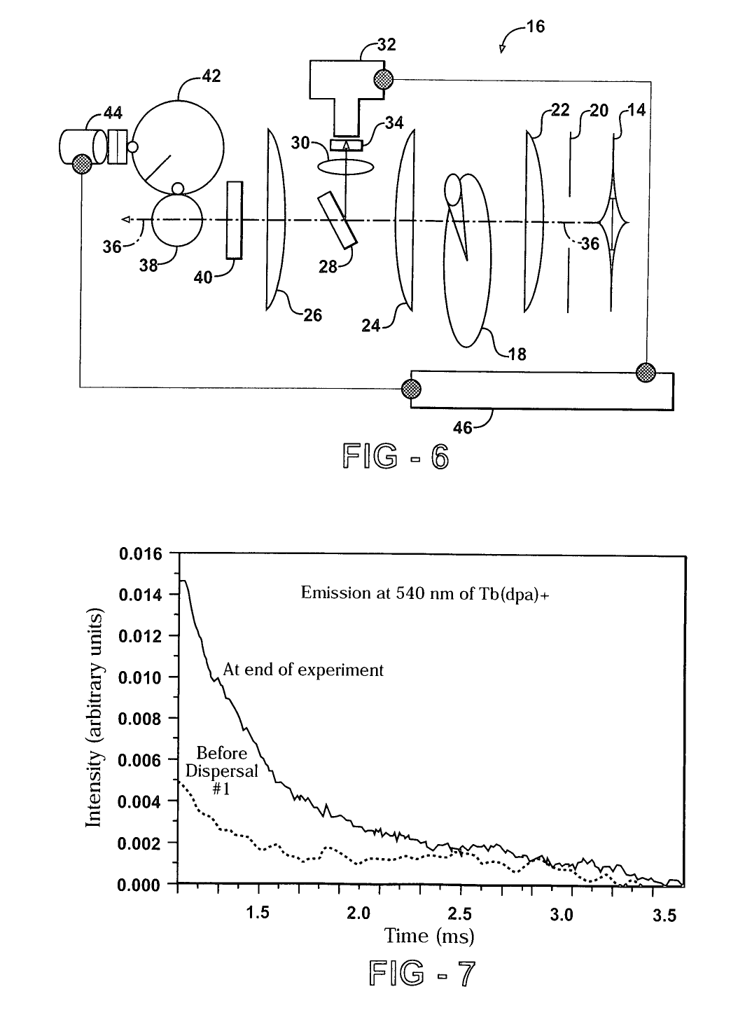

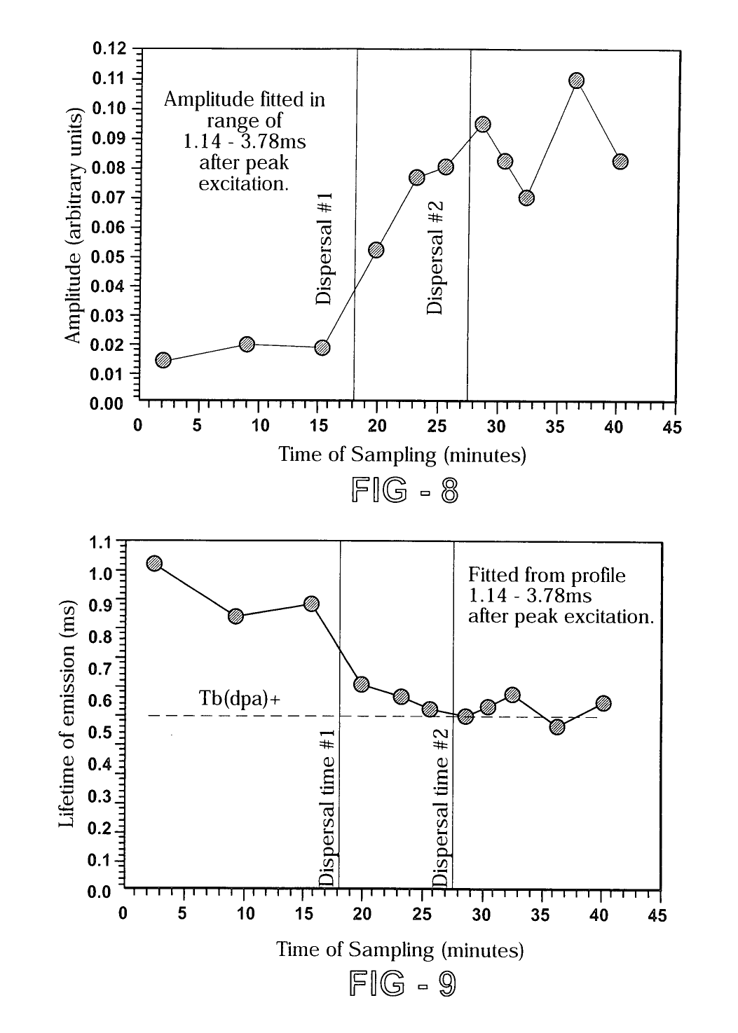

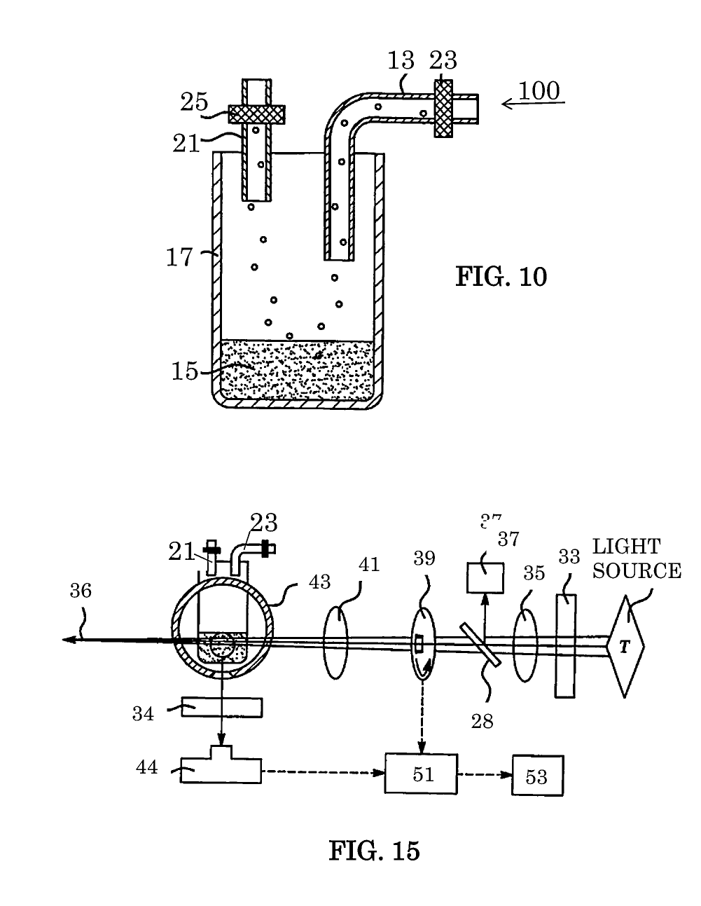

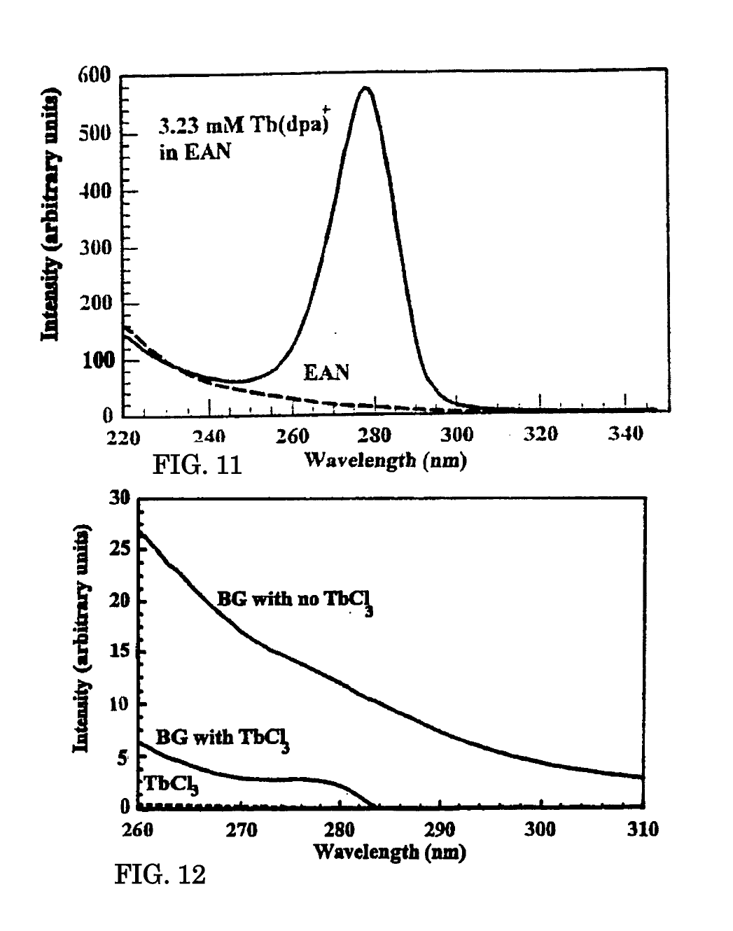

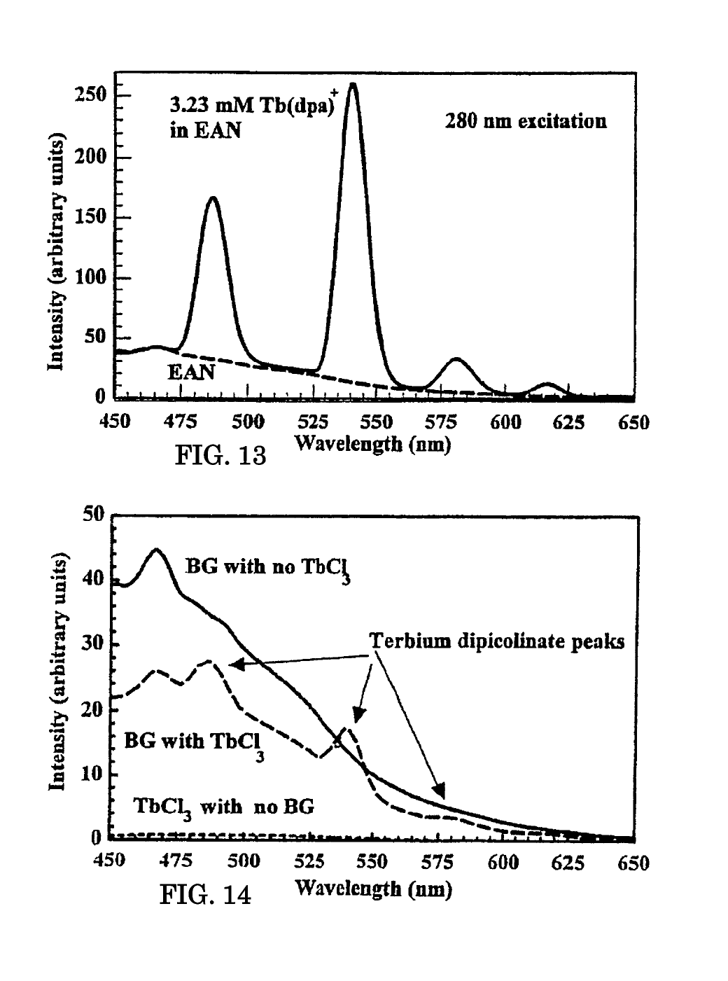

The method of detection for airborne bacterial endospores is demonstrated by the experimental results shown in FIG. 7. A terbium salt solution was illuminated by a series of UV pulses that occurred over 41 minutes, and photoluminescence time profiles determined for separate UV pulses. Aerosol particles were continuously sampled in the atmosphere of an aerosol chamber over the entire time. Bacterial endospores were released into the aerosol chamber by a dispersal device at a time of 18 minutes, and the empty dispersal device turned on a second time at 27 minutes. FIG. 7 shows two photoluminescence time profiles: a profile before the 18 minutes (i.e., before dispersal #1) and a profile at the end of 41 minutes (i.e., the end of the experiment). The difference between the two profiles is an indication that bacterial endospores were actually collected sometime during the experiment.

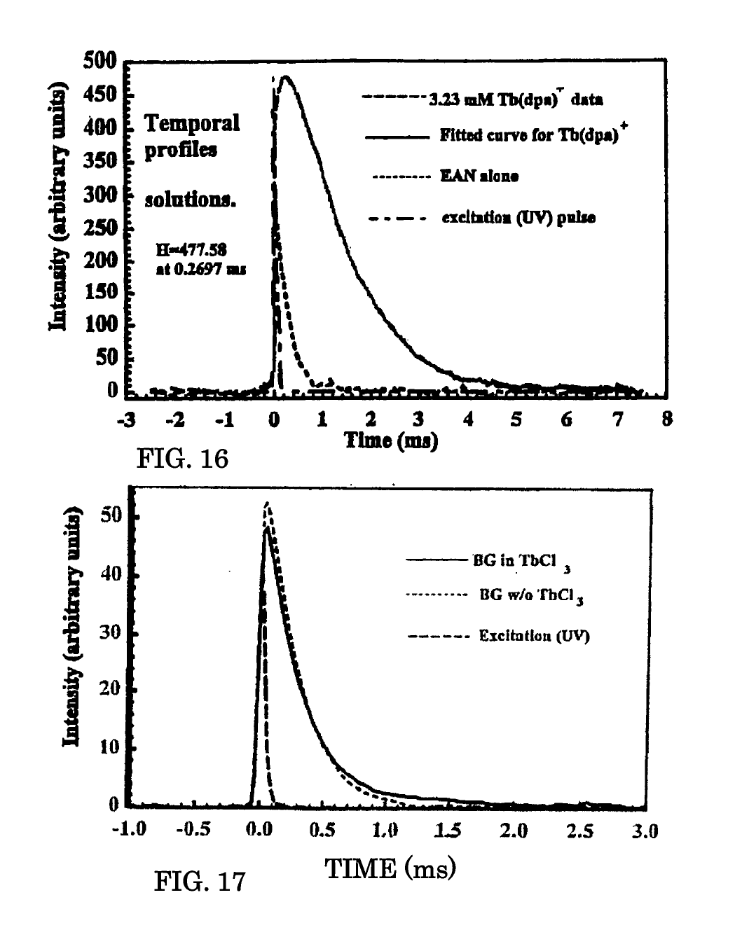

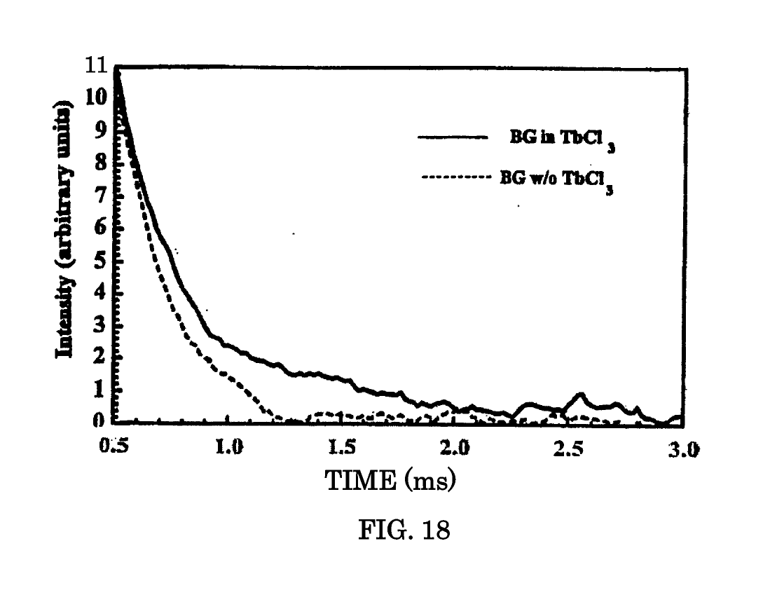

The continuous use of the detector was demonstrated in an experiment, the results of which are shown in FIGS. 8 and 9. Aerosols were collected from the atmosphere of an aerosol chamber continuously for 45 minutes by the device, while the photoluminescence emission profiles from the collection vessel were determined by the device from a series of repeated UV excitation pulses. Bacterial endospores were released into the atmosphere at Dispersal Time #1. The signal from each pulse is shown in FIG. 8, and the photoluminescence lifetime determined for each pulse is shown in FIG. 9. Although the collection of aerosols did not stop for 45 minutes, the sudden change in both photoluminescence signal and photoluminescence lifetime is easily seen at Dispersal Time #1.

In the detection method, if the emission intensity at wavelengths distinctive of the lanthanide chelate is significantly above a threshold level, endospore content is determined to be present in the sample medium. On the other hand, if the emission intensity at the distinctive emission wavelength(s) of the lanthanide chelate is relatively low, for example, close to the threshold level, the lanthanide chelate, and hence bacterial endospores, are determined not to be significantly present in the sample medium. After a short delay from the end of the excitation pulse, a photodetector can be used to determine whether any luminescence exists that is indicative of the presence of lanthanide chelate, and hence endospores. Because most substances have luminescence lifetimes on the order of nanoseconds whereas lanthanide chelates such as terbium dipicolinate and europium dipicolinate have relatively long luminescence lifetimes (2.0 milliseconds and 0.8 milliseconds, respectively), the presence of any luminescence after several nanoseconds is highly indicative of the presence of the lanthanide chelate and thus also endospores. On the other hand, if no significant luminescence is present after several nanoseconds, the sample medium is determined to contain no significant endospore content.

FIG. 8 shows the amplitude of the emitted light as a function of time. FIG. 9 shows the photoluminescence lifetime changing as the impinger collects endospores. The lifetime of the suspension after dispersion approached the lifetime of terbium dipicolinate thereby indicating the presence of endospores in the sample.

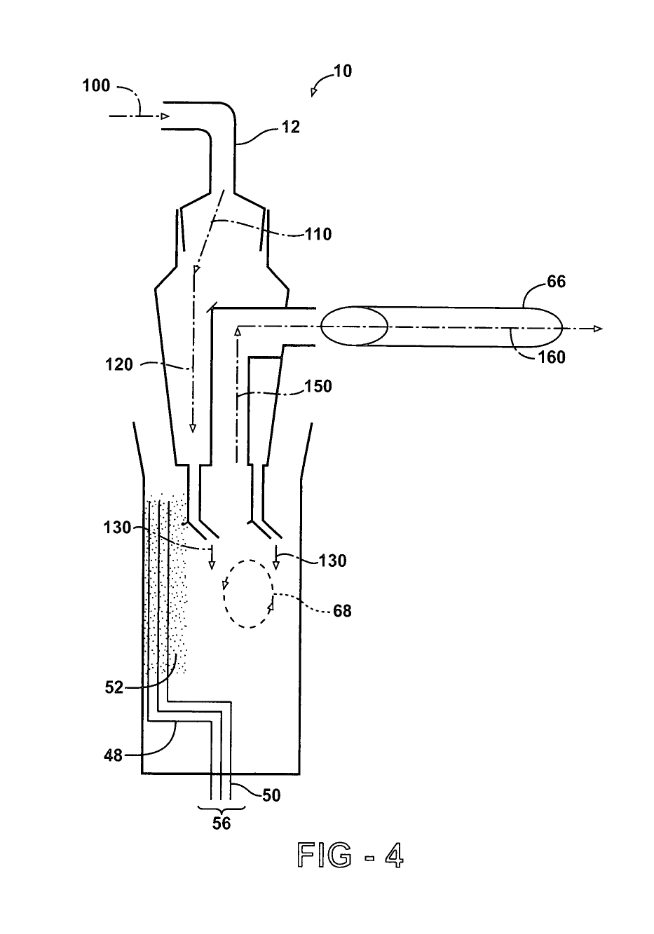

In practice the optical apparatus 16 can include the ultraviolet beam 36 propagating in free space. In another embodiment, however, the optical set-up 16 may include the ultraviolet beam 36 transmitted through optical fibers 48, 50 as shown in FIGS. 2 and 4. The two fibers may be fastened or twisted together to form a single dual fiber separated by a cladding or a jacket. The two fibers coming together are shown in FIG. 2. The dual fiber, which is formed by fastening or twisting 48 and 50 together, is labeled 56 in FIG. 4.

This optical set-up has a first optical fiber 48 that transmits ultraviolet radiation to excite the lanthanide solution and a second optical fiber 50 that collects photoluminescence generated in the lanthanide salt solution. In this embodiment, the ultraviolet light travels through a quartz optical fiber that is inserted into the collection vessel. The ultraviolet light leaves the fiber and excites the contents. A glass or quartz optical fiber with collection optics then collects the emitted light. The fibers may selectively be entered through a bottom center portion of the collection vessel 38 as shown in FIGS. 3 and 4 in order to avoid disturbing flow, however fibers may be entered at other selectable positions.

FIGS. 3 and 4 show still another embodiment in which the aerosol concentrator 12 contains a sol-gel 52 having a liquid phase containing the lanthanide salt solution. In this embodiment, the optical set-up 16 may include an integrating sphere 42 having an aperture positioned to collect photoluminescence from the lanthanide salt solution, as shown in FIG. 3. In another version of this embodiment, shown in FIG. 4, a front and backscatter probe 56 utilizes a first optical fiber 48 used to transmit ultraviolet radiation that excites the sol-gel 52 and a second optical fiber 50 used to collect photoluminescence emitted from the sol-gel 52 upon excitation of the sol-gel 52.

A system is also provided for locating an aerosol particle that contains bacterial endospores. The system, shown in FIG. 5, is comprised of a sol-gel 52 with a lanthanide salt dissolved in a liquid phase, an active aerosol collector 12 in the form of an impactor for depositing the aerosol particle on the sol-gel 52, a light source 14 for transmitting an ultraviolet light through a broadband filter 34 on to the sol-gel 52 and an imager 54. In a particular version of this embodiment, a microscope 58 is used in conjunction with a narrowband filter 60 to focus the imager 54 on the sol-gel 52 in order to distinguish an endospore aerosol 62 from a nonspore aerosol 64. In another embodiment, the imager 54 is a low-light camera.

A method is further provided for detecting bacterial endospores suspended in the atmosphere. The method is comprised of the steps of: (a) collecting an air sample from the atmosphere over a given period of time; (b) generating a plurality of aerosol samples from said air sample in an aerosol concentrator 12; (c) combining at least one of the aerosol samples with a lanthanide solution; (d) illuminating the lanthanide solution with a light source 14; (e) collecting the photoluminescence emitted by the lanthanide solution; and (f) calculating the amplitude and lifetime of the luminescence emitted by the lanthanide solution. The step of measuring the temporal profile of the collected aerosols may selectively be performed and as an alternative to step (f), the luminescence that occurs after a delay may be time integrated. As an alternative to step (f), the luminescence that occurs after a delay may be time integrated.

While the principles of the invention have been shown and described in connection with but a few embodiments, it is to be understood clearly that such embodiments are by way of example and are not limiting. Furthermore, all publications and patents cited in the specification are incorporated herein by reference.

1. A device to detect bacterial endospores suspended in a volume of atmosphere comprising:
an active aerosol concentrator actively collecting the volume of atmosphere:
a collection vessel operatively associated with the active aerosol concentrator for receiving the volume of atmosphere; the collection vessel containing a lanthanide salt solution that mixes with the volume of atmosphere and produces a photoluminescent lanthanide dipicolinate complex in the presence of bacterial endospores;
an excitation energy source for providing excitation energy in the form of a pulse for excitation of the lanthanide salt solution within the collection vessel to produce photoluminescence of the lanthanide dipicolinate complex lasting for a longer period of time than the photoluminescence of the lanthanide salt solution;
a decay time detector for detecting the decay time of the photoluminescence within the collection vessel of one of the lanthananide salt solution or the lanthanide dipicolinate complex;
the decay time detector operating to measure the time of decay of the photoluminescence and operating to generate a positive response only after the decay time of the photoluminescence of the lanthanide salt solution has subsided;
whereby if bacterial endospores are present in the volume of atmosphere a lanthanide dipicolinate complex will be produced and after the photoluminescence of the lanthanide salt solution has subsided, the photoluminescence of the lanthanide dipicolinate complex will be detected by the decay time detector.

2. The device of claim 1, wherein the aerosol concentrator is selected from the group consisting of a bubbler, an impinger, an impactor and combinations thereof.

3. The device of claim 1, wherein the lanthanide salt solution comprises trivalent terbium cations, the terbium cations emitting phosphorescence at a predetermined level for a first time interval that is less than one ms after the pulse and the lanthanide dipicolinate complex emitting phosphorescence at a predetermined level for a second, longer time interval in excess of one ms, and wherein the detection of photoluminescence occurs after the first duration of time and before the end of the second time interval so that the phosphorescence is substantially limited to that of the lanthanide dipicolinate complex.

4. The device of claim 1 wherein the lanthanide salt solution comprises europium cations.

5. The device of claim 1, wherein the excitation energy source comprises an ultraviolet light that is pulsed and wherein the decay time detector operates to detect photoluminescence several milliseconds after the end of a pulse.

6. The device of claim 1, further comprising a chopper which creates a pulse and wherein the decay time detector operates to detect photoluminescence within a range of approximately 1.14 to 3.78 milliseconds after the end of a pulse.

7. The device of claim 1, further comprising an electronic modulator which modulates the excitation energy source and wherein the decay time a detector operates to detect photoluminescence within a range of approximately 1.14 to 3.78 milliseconds after the end of the pulse.

8. A device to detect bacterial endospores suspended in a volume of atmosphere comprising:
an excitation energy source, said source providing excitation energy in the form of an ultraviolet light;
an aperture proximate said ultraviolet light, said aperture affording an ultraviolet light beam from said ultraviolet light;
a first focusing dement downstream of said aperture, said first element focusing said ultraviolet light beam before said beam reaches a silica window;
a second focusing element downstream of said silica window, said second element focusing said ultraviolet light beam after passing through said silica window and before reaching a collection vessel;
an active aerosol concentrator fluidly connected to said collection vessel actively collecting the volume of atmosphere;
said collection vessel containing a lanthanide salt solution that yields a photoluminescent lanthanide dipicolinate complex in the presence of bacterial endospores; and
a photomultiplier downstream of said collection vessel, said photomultiplier collecting photoluminescence associated with said complex detecting the bacterial endospores suspended in the volume of atmosphere.

9. (canceled)

10. (canceled)

11. The device of claim 8, further comprising an integrating sphere located between said collection vessel and said photomultiplier.

12. The device of claim 8. wherein said silica window reflects at least part of said ultraviolet light beam.

13. (canceled)

14. (canceled)

15. (cancelled)

16. The device of claim 1, further comprising an optical fiber for transmitting said excitation energy.

17. The device of claim 16, wherein said optical fiber transmits said excitation energy to said lanthanide solution and further comprising a second optical fiber collecting photoluminescence generated by said lanthanide salt solution.

18. The device of claim 1, wherein said collection vessel contains a sol-gel having a liquid phase containing said lanthanide salt solution.

19. The device of claim 18 further comprising:
a first optical fiber transmitting ultraviolet light to said sol-gel; and
a second optical fiber collecting photoluminescence generated from said sol-gel having a liquid phase containing said lanthanide salt solution.

20. A device for locating an aerosol particle that contains bacterial endospores comprising:
an active aerosol concentrator, said concentrator including an impactor;
a container having a sol-gel with a lanthanide salt dissolved in a liquid phase;
a light source, said source transmitting an ultraviolet light through a broadband filter onto said sol-gel; and
an imager, for the purpose of imaging an aerosol particle when said impactor affords said particle to impact said sol-gel.

21. The device of claim 1, wherein the lanthanide salt solution is selected from the group consisting of a polar solvent and a room temperature ionic liquid.

22. The device of claim wherein the lanthanide salt solution comprises trivalent terbium cations that are adapted to combine with spore-specific “target” molecules to form a photoluminescent lanthanide dipicolinate complex with an increased duration of phosphorescence that can be detected by the decay time detector following a predetermined time delay after the pulsed excitation energy occurs, and whereby the phosphorescence of the photoluminescent lanthanide dipicolinate complex is distinguishable from the phosphorescence of the trivalent terbium cations based upon its longevity.

23. The device of claim 22 wherein the decay time detector further comprises an electronic oscilloscope for recording the phosphorescence of the lanthanide dipicolinate complex within the collection vessel and wherein the dissolved lanthanide salt comprises trivalent terbium cations that are adapted to combine with bacterial endospores to form a photoluminescent lanthanide dipicolinate complex with an increased duration of phosphorescence that can be detected by the electronic oscilloscope after the pulse occurs, and whereby the phosphorescence of the photoluminescent lanthanide dipicolinate complex is distinguishable from the phosphorescence of the trivalent terbium cations based upon its longevity.

24. The device of claim 23 wherein the decay time detector operates to detect photoluminescence in the range of approximately 1 millisecond to 4 milliseconds after the pulse which effectively distinguishes photoluminescence arising from the lanthanide dipicolinate complex versus the photoluminescence of the lanthanide salt solution.

25. The device of claim 23 wherein the decay time detector operates to detect photoluminescence approximately 1.14 milliseconds after the pulse, which operatively distinguishes photoluminescence arising from the lanthanide dipicolinate complex from the photoluminescence of the lanthanide salt solution.
